(19) United States
(12) Patent Application Publication
(10) Pub. No.: US 2007/0003497 A1
(43) Pub. Date: Jan. 4, 2007

(54) Device and method for mixing liquids and oils or particulate solids and mixtures generated therefrom

(76) Inventors: William D. Holloway JR., Carlsbad, CA (US); Michael A. Holloway, Escondido, CA (US); Kenneth H. Tarbet, Oceanside, CA (US)

Correspondence Address: PROCOPIO, CORY, HARGREAVES & SAVITCH LLP, 530 B STREET, SUITE 2100, SAN DIEGO, CA, 92101, US

(21) Appl. No.: 11/448,602

(22) Filed: Jun. 6, 2006

Related U.S. Application Data
(63) Continuation-in-part of application No. 11/302,967, filed on Dec. 13, 2005.
(63) Continuation-in-part of application No. 10/420,280, filed on Apr. 21, 2003.
Application No. 10/420,280 is a continuation-in-part of application No. 10/301,416, filed on Nov. 21, 2002.
Application No. 10/301,416 is a continuation-in-part of application No. 09/698,537, filed on Oct. 26, 2000, now Pat. No. 6,521,248.
(60) Provisional application No. 60/595,095, filed on Jun. 6, 2005.
(60) Provisional application No. 60/596,170, filed on Sep. 6, 2005.
(60) Provisional application No. 60/780,947, filed on Mar. 8, 2006.
(60) Provisional application No. 60/801,231, filed on May 16, 2006.
(60) Provisional application No. 60/635,915, filed on Dec. 13, 2004.
(60) Provisional application No. 60/596,170, filed on Sep. 6, 2005.
(60) Provisional application No. 60/594,612, filed on Apr. 22, 2005.
(60) Provisional application No. 60/594,540, filed on Apr. 15, 2005.
(60) Provisional application No. 60/161,546, filed on Oct. 26, 1999.

(51) Int. Cl. A61K 8/00

(52) U.S. Cl. 424/59

(57) ABSTRACT

A cavitation device includes a pair of axially aligned opposing nozzles within a housing. Liquid is introduced into the nozzles at a pressure to create rotational vortices within the nozzles, causing cavitation. The thermo-physical reactions resulting from cavitation produce an increase in heat and breaking of the bonds holding large fluid arrays together. Additional cavitation is induced by a collision between the liquid outputs of the opposing nozzles to enhance mixing. By subjecting a mixture of material(s) to be dissolved in water to a cavitation device, a true solution of a lipophilic and water or stable suspension of particulate solids in water can be formed.

### CROSS-REFERENCE TO RELATED APPLICATIONS

This application claims priority to each of provisional application No. 60/595,095, filed Jun. 6, 2005, No. 60/596,170, filed Sep. 6, 2005, No. 60/780,947, filed Mar. 8, 2006, and No. 60/801,231, filed May 16, 2006, and is a continuation-in-part of application Ser. No. 11/302,967, filed Dec. 13, 2005, which claims the priority to provisional applications No. 60/635,915, filed Dec. 13, 2004, No. 60/596,170, filed Sept. 6, 2005, No. 60/594,612, filed Apr. 22, 2005 and No. 60/594,540, filed Apr. 15, 2005, and which is a continuation-in-part of Ser. No. 10/420,280, filed Apr. 21, 2003; which is a continuation-in-part of application Ser. No. 10/301,416, filed Nov. 21, 2002, which is a continuation-in-part of application Ser. No. 09/698,537, filed Oct. 26, 2000, now issued as U.S. Pat. No. 6,521,248, which claims priority to provisional application No. 60/161,546, filed Oct. 26, 1999. Each of the above-identified applications is incorporated by reference in its entirety and for all purposes.

### BACKGROUND OF THE INVENTION

All liquids are made of molecules that interact in a system of attraction in equilibrium with repulsions. These forces play an important role in the formation of large molecular matrices or arrays or pseudo-polymeric systems. Such large arrays or pseudo-polymeric structures are responsible for many of the liquids observed properties, such as boiling point, surface tension and viscosity, for example. The disruption of these large molecular associations or pseudo-polymeric interactions results in modulation of the liquids properties.

Common knowledge has it that oil and water do not mix. Oil-like liquids, called “lipophilic”, have historically been categorized as hydrophobic, having no miscibility in water. Substantial research has been dedicated to the search for methods and techniques whereby a stable emulsion can be made of hydrophobes and hydrophiles, e.g., pulling oils and lipophilics into solution in water. Water is a polar molecule, and hydrophiles are water loving due to one or more polar interactions. These polar interactions often involve a hydrogen atom, which is bound to a polarizable atom, such as oxygen, an interaction that is often referred to as hydrogen bonding. Hydrogen bonding is one key interaction that impacts the solubility of a substance in water. Most hydrophiles, such as sugar, table salt and even drinking alcohol are able to form hydrogen bonds with water, and thus are soluble.

Hydrophobes, such as oils are a large class of compounds and compositions that are not able to form hydrogen bonds with water. Moreover, many oils are non-polar, meaning the molecule does not have charged regions. In general, hydrophobes are not water-soluble secondary to the inability to form hydrogen bonds, which is related to the absence of charged regions.

The cosmetic industry has expended substantial research in the field of surfactants to enhance the solubility of hydrophobes in water. Most surfactants are man-made chemicals designed to have a both a hydrophilic portion and a hydrophobic portion, in which the hydrophilic “head” is soluble in water while the hydrophobic “tail” is soluble in the oil. The surfactant is able to form an emulsion or suspension of small globules of oil in water, with the surfactant acting as the bridge between the oil and the water. This type of system can be undesirable from several perspectives, not the least of which are toxicity, skin irritation and manufacturing costs.

Another approach has involved suspending lipophilics as nanoparticles, in water. However, the terms suspension and solvation are dissimilar. Suspension relates to the suspension of a pure particle-like substance in the suspending agent. For example, smoke from a fire is a suspension in the air, as the smoke particles are not soluble in the air, yet are light enough that they are kept aloft in the suspending medium, which is the air, for a period of time. The period of suspension is directly related to the viscosity of the suspending medium and the size and density of the suspended particle. Nonetheless, regardless of size, the particles of a suspension can be recovered through centrifugation and/or filtration.

Solvation and the miscibility of liquids stand in stark contrast to suspension. When a solid is dissolved in a solvent, the solid is broken down to individual molecules that are dispersed throughout the solvent. A similar dispersion at the molecular level occurs when one liquid dissolves another. When a liquid is soluble in another liquid, the two liquids are said to be miscible. Just like solubility, miscibility is a function of temperature and is specific to each solute and solvent. A two-layered system results where the liquids are immiscible or only partially miscible. In the case of oils and/or lipophilics whose miscibility with water has historically been defined at 0.0, there are very few exceptions. For example, olive oil is given the “completely insoluble at all temperatures and pressures” designation in the 60th edition of the CRC Handbook of Chemistry and Physics, published by CRC Press.

In addition to the mixing of oils and/or lipophilics, there is continually a need for processes whereby particleized solids can be dissolved or stably suspended in fluids. Each of the foregoing needs is addressed by the present invention.

### BRIEF SUMMARY OF THE INVENTION

It is an advantage of the present invention to provide a device and method for processing liquids to dissolve components in water that are traditionally considered to be insoluble.

It is another advantage of the present invention to provide compositions of water and dissolved components created using the inventive device and method for cosmetic and therapeutic applications and for use in foods and manufacturing industries.

According to the present invention, by subjecting a mixture of the material(s) to be dissolved and water to a vortexing assembly such as that disclosed in U.S. Pat. No. 6,521,248, a true solution or stable suspension of the material can be formed. The vortexing assembly pressurizes a starting fluid to a first pressure followed by rapid depressurization to a second pressure to create a partial vacuum pressure that results in the formation of cavitation bubbles that subsequently implode when they encounter a higher pressure. The thermo-physical reactions provided by the implosion of the cavitation bubbles result in an increase in heat and breaking of bonds holding large fluid arrays together. This process can be repeated until a desired physical-chemical trait of the fluid mixture is obtained.

In the preferred embodiment a plurality of rotational vortices, also referred to as nozzles, are used. The nozzles are enclosed within a housing includes an inlet corresponding to each nozzle and an outlet for discharging the mixed fluid. The liquid is introduced through openings in the sides of the nozzles to spin the liquid through one 360 degree rotation, then discharge the liquid through an exit opening at the radial center of the nozzle. The nozzle outlets are each directed into an exit volume in an arrangement so that the output stream from each of the nozzles collides with the other outputs. This high energy collision results in generation of additional cavitational energy and mixing of the liquid. It is therefore preferred that the nozzles are axially aligned, with two outlets directly opposing each other. In the exemplary embodiment, the housing is tubular in shape so that two opposing nozzles are axially aligned with the housing. In the preferred embodiment, the separation between the nozzles is made variable by mounting the nozzles on sliding attachments to permit adjustment of the desired interaction based upon liquid viscosity or nature of the component(s) to be mixed.

In addition to the use of rotational vortices, other techniques known to those of skill in the art can be used to create cavitation in a fluid so long as the cavitating source is suitable to generate sufficient acoustic energy to break the large arrays. The acoustic energy produced by the cavitation provides energy to break the large fluid arrays into smaller fluid clusters. For example, acoustical transducers may be utilized to provide the required cavitation source. In addition, a fluid may be forced through a tube having a constriction in its length providing for a high pressure before the constriction, which is rapidly depressurized within the constriction and then pressurized again after the restriction. Another example includes forcing a fluid in reverse direction through a volumetric pump.

In a preferred embodiment of the inventive method, water that has previously been fractionated using the cavitation device is added to the device and the device is activated. The material to be mixed, e.g., oil, is added to the water in the device. The water and material are processed through the device for a specified time, which preferably involves multiple cycles through the device.

A mixture containing lipophilic and hydrophilic components, which may exist as a two or more layer system, can be subjected to the cavitation device resulting in at least partial solvation of one or more lipophilic members into the hydrophilic component. For example, in conventional processing, a mixture of 10% olive oil in water will be a two phase system, with no oil dissolved in the water. However, subjecting this two phase system to the physics device will result in the solvation of about 0.04% olive oil in the water. This invention is not limited to dissolving oil in water, yet shall include dissolving of any lipophilic liquid into the water.

In another aspect of the invention, the dissolving of oil in water facilitates the dissolving of additional lipophilic substances into the water. In one embodiment, a lipophilic drug may be more soluble in the oil in water system, thereby facilitating the incorporation of lipophilic drugs into a water based delivery system without the need of surfactants or other solvation enhancers.

### BRIEF DESCRIPTION OF THE DRAWINGS

FIG. 1 is a front elevation of the cavitation device showing the placement of the nozzles within the housing.

FIG. 2 is a diagrammatic view of a system for mixing incorporating the cavitation device.

FIG. 3 is an exploded side elevation of a nozzle for use in the inventive device.

FIG. 4 is an entrance face view of the front section of the nozzle of FIG. 3.

FIG. 5 is an interior face view of the back section of the nozzle of FIG. 3.

FIGS. 6 are an entrance face view of the vacuum plate of the nozzle of FIG. 3.

FIG. 7 is a diagrammatic view of the exit orifice of the nozzle showing the spray pattern of the exiting liquid.

FIG. 8 is a diagrammatic top view of an alternate embodiment of the device with two sets of nozzles.

FIG. 9 is a diagram of a micelle-like assembly resulting from inclusion of phosphatidyl choline (lecithin) in a micro emulsion.

FIG. 10 is a plot of backscattering intensity versus height in a mixture of safflower oil, macadamia nut oil, borage oil and lecithin in water at different times.

FIG. 11 is a plot showing distribution of particle diameter in a mixture of 10% olive oil and lecithin in water.

FIG. 12 is a pair of plots generated during stability analysis of jojoba oil particles, with the upper plot showing backscattering intensity versus time and the lower plot showing distribution of particle diameter, in a mixture of jojoba oil and lecithin in water.

FIG. 13 is a plot showing distribution of particle diameter, in a mixture of jojoba oil and lecithin in water.

FIG. 14 is a pair of plots generated during stability analysis of tea tree oil particles, with the upper plot showing backscattering intensity versus time and the lower plot showing distribution of particle diameter, in a mixture of tea tree oil and lecithin in water.

FIG. 15 is a plot of backscattering intensity versus height in a mixture of tea tree oil and lecithin mixed in water at different times.

### DETAILED DESCRIPTION OF THE INVENTION

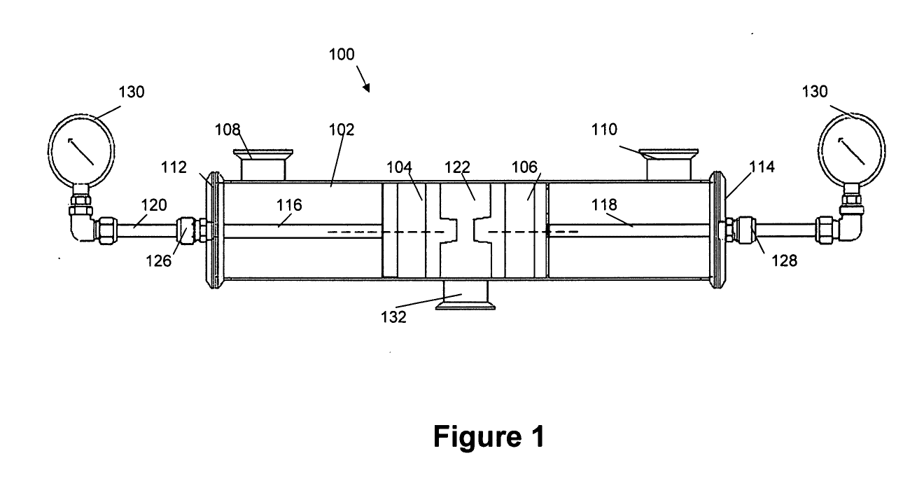

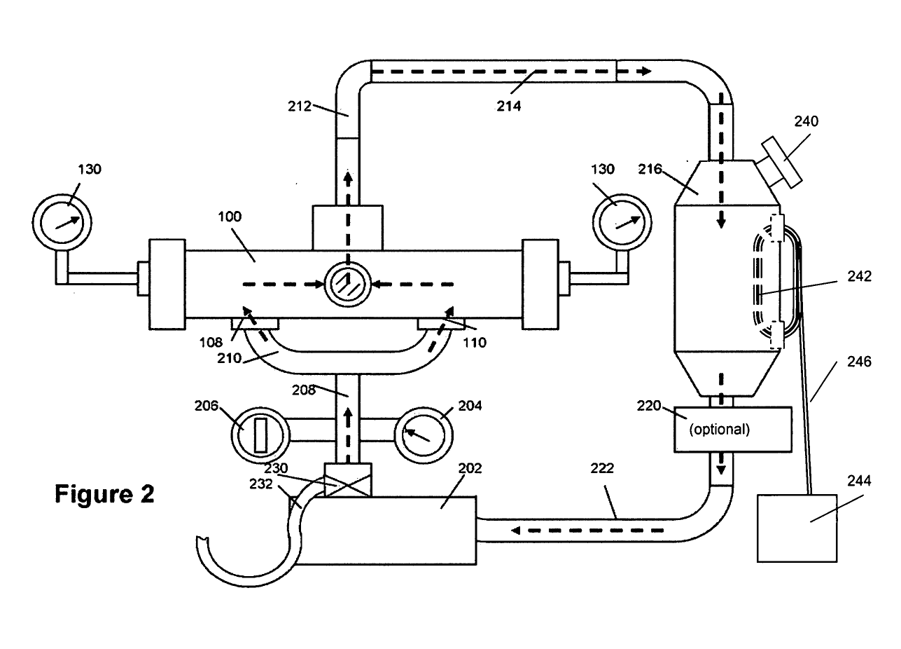

An exemplary embodiment of the inventive cavitation device 100 is illustrated in FIG. 1. The device 100 has a tubular housing 102 which encloses a pair of nozzles 104 & 106. Housing 102 is preferably formed from 316 stainless steel tubing or a similar corrosion resistant, inert material that is capable of withstanding the elevated operating pressures required for practicing the cavitation process. In the exemplary embodiment, housing 102 has a diameter on the order of 60 to 80 mm (2.4 to 3.2 in.), although other dimensions may be selection for different applications. End caps 112 and 114 are attached to opposite ends of housing 102 using a pressure-resistant seal. Liquids are introduced through inlet ports 108 and 110, where port 108 supplies nozzle 104 with liquid and port 110 is the supply for nozzle 106. The liquid entering through the two inlet ports is forced into the backside of the corresponding nozzle through a tangential channel and through the nozzle orifice. The nozzles 104 and 106 are oriented in an axially aligned, opposing relationship so that the liquid output from each nozzle will directly collide with the output from the other nozzle. This high energy collision results in generation of additional cavitational energy and mixing of the liquid. The nozzles 104, 106 emit liquid into common exit volume 122 and the liquid passes out of the device through discharge port 132. A view port (shown in FIG. 2) may be provided in housing 102 adjacent to the exit volume 122 to permit observation of the fluid during cavitation.

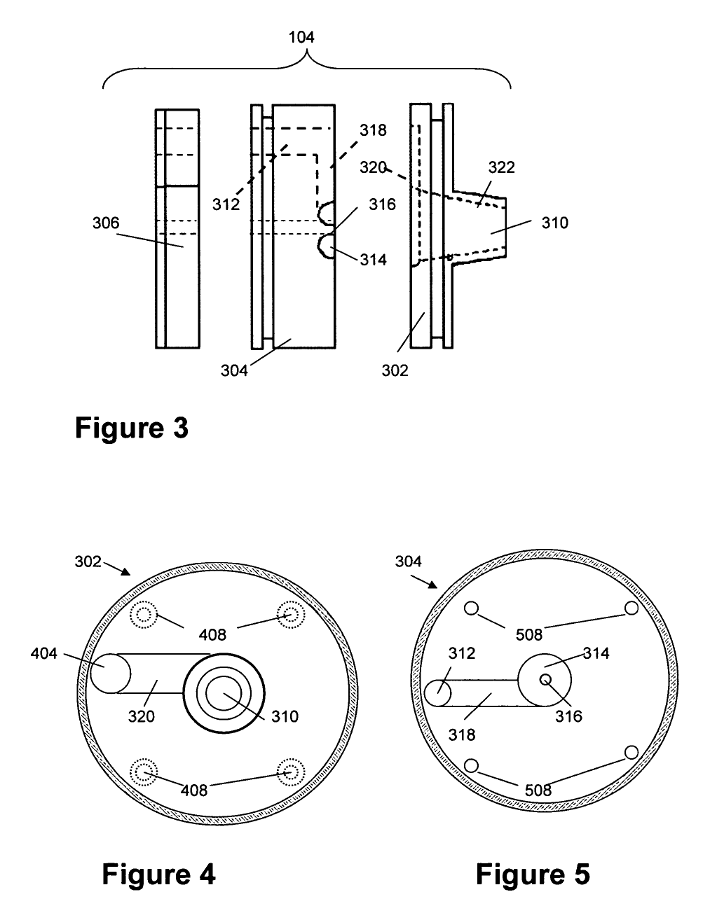

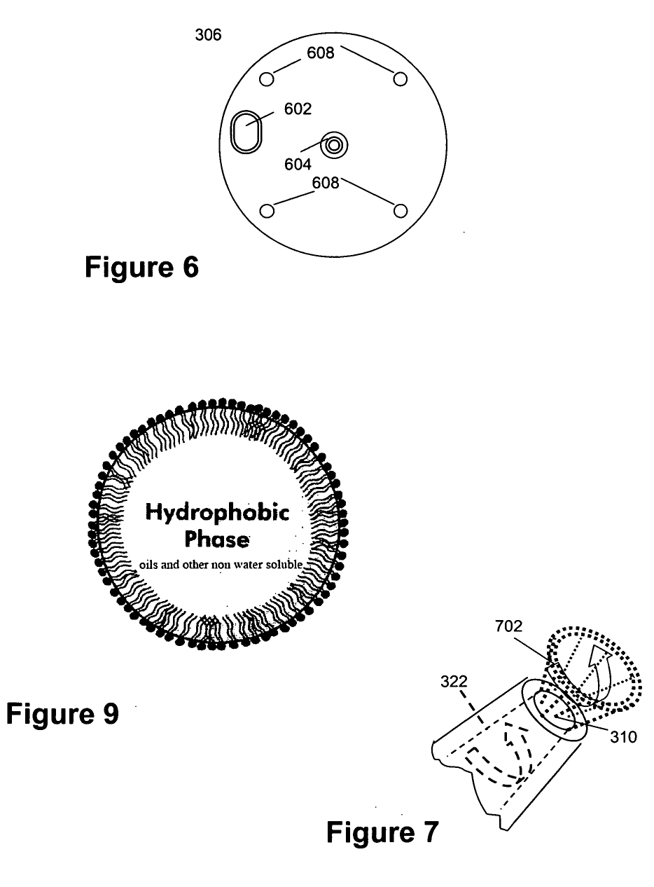

Details of the nozzle construction are illustrated in FIGS. 3-6. Nozzle 104 is illustrated in FIG. 3. Nozzle 106 is identical in construction to nozzle 104 but it oriented within housing 102 as a mirror image to nozzle 104. Each nozzle includes three sections, the front section 302, through which the liquids exit, the back section 304, which combines with section 302 to create the rotational vortex needed to induce cavitation, and vacuum plate 306, which seals the entrance side of the nozzle within the housing interior so that all liquids are forced through the nozzle opening. In the preferred embodiment, the front and back sections are formed from Teflon® (polytetrafluoroethylene) and the vacuum plate 306 is formed from 316 stainless steel.

Front section 302 includes a tapered cone that includes exit orifice 310. FIG. 4 illustrates the inlet side of front section 302, which, when assembled with back section 304, shown in FIG. 5, provides a whirl chamber which is tangentially fed by the feed tube formed by combining recessed channels 320 and 318 of the front and back sections respectively. The whirl chamber is formed from the combination of circular channel 314 and conical surface 322, with raised center through which vacuum port 316 extends to define a donut that ensures that the liquid is directed to the sidewalls of conical surface 322 to generate the desired vortex.

Vacuum plate 306 has an opening 602 through which liquids enter the nozzle. Opening 602 is aligned with input opening 312 in back section 304. Bores 408, 508 and 608 are aligned to permit screws (not shown) to be inserted from the exit side of front section 302 (where bores 408 are countersunk) to be screwed into bores 608, which are threaded to receive the screws.

Vacuum plate 306 preferably has a compressible O-ring seal such as silicone or Viton® around its circumference to provide a tight seal between the edges of plate 306 and the inner surface of housing 102 while allowing the position of the nozzle to be moved axially within the housing. An additional aspect of the present invention relates to the ability to alter the distance between the nozzles 104, 106 as needed to achieve a desired interaction. The optimal distance may be specific to liquid viscosity and/or may relate to solid components of the liquid, such as in a suspension type system. The optimal distance may be further dictated by optimal treatment temperature per mixture/liquid to be treated. The optimal distance may further be correlated by atmospheric conditions. The provide for such needs, the nozzles of the present device are adjustably connected within the outer housing by means of steel tubes 116, 118 that are slidably inserted through the endcaps 112, 114 of the housing 102 and attached to the vacuum plates 306 at center vacuum orifice 604 (on the order of 1.6 mm ( 1/16^{th} in.)), allowing the distance between the nozzles 104, 106 to be adjusted to a particular need, such as viscosity of the liquid to be processed. Once the desired separation between the nozzles is achieved, their positions are fixed in place by tightening a Swagelock® 126, 128 or similar fastener attached to each endcap 112, 114. Appropriate fasteners and materials for providing the adjustable nozzle separation are known in the art. Vacuum gauges 130 connected to each tube 116, 118 measure the vacuum produced at the rotational vortex within each nozzle through vacuum orifices 604 and 316. The vacuum orifices also provide means for introduction of liquids to be mixed by way of a cannula and an appropriate T-connection (not shown), which is generally known in the art.

As the liquid is forced through the rotational vortex, centripetal and centrifugal forces cause the water to take on laminar flow and to be forced against the outer portion of the tube through which the liquid is being forced. This combination of forces actually produces laminar flow liquid that is simultaneously rotating. However, this laminar flow liquid is different than the normal understanding of laminar flow fluids. The water flowing from a standard garden hose, is one embodiment of well known laminar flow. However, in the garden hose type of laminar flow the water is of singular molecular motion, in the direction of exiting the hose. Moreover, the water from the garden hose will mimic the interior shape of the hose after exiting the house, until the flow energy is dissipated. However, in the present system, the liquid is forced into a rotational vortex in two dimensions, such that the molecules are rotating in the same rotational manner as the vortex through which the liquid was forced. Secondly, according to the pressure exerted by the liquid being forced around the radius of curvature and the resultant centripetal and centrifugal forces exerted on the molecules of the liquid, the molecules are coerced into a rotational motion simultaneous with being coerced into a laminar flow situation. However, unlike the garden hose example, because the liquid is being forced against the wall of the passage while being coerced into a rotational motion, when the liquid exits the nozzle and is released from the confining tube of the rotational vortex, the liquid forms a thin sheet of liquid. FIG. 7 illustrates the effect that the whirl chamber and conical surface 322 have on the output stream of liquid 702. The liquid emitted from exit orifice 310 has a hollow cone spray pattern that rotates in the same direction with which it was introduced into the whirl chamber. Each nozzle 104 and 106 emits the same spray pattern. For further maximizing the effect of the collision of the output streams, the cone spray patterns can rotate in opposite directions. The resulting outputs of the nozzles have rotational momentum and uniform outwardly radiating force, describing a parabola with the vertex at the exit point of the nozzle.

The interior diameter of the feed channel through which the liquid passes, as well as the diameter of the nozzle exit orifice may be altered in size to accommodate need and desired outcome.

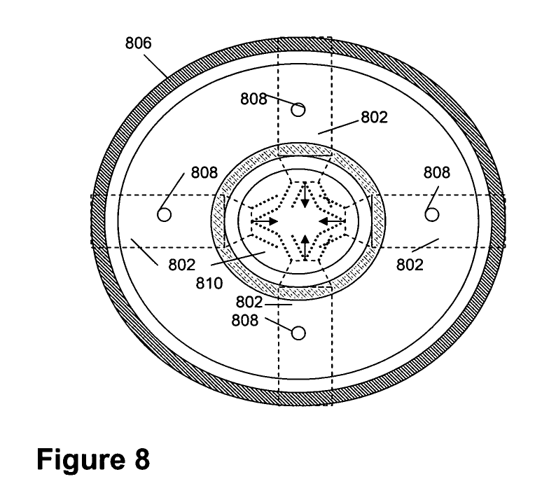

An alternate embodiment of the cavitation device is illustrated in FIG. 8. Four small inverted pump volutes (nozzles) 802 made of Teflon® (without impellers are housed in a 316 stainless steel pipe housing 806. The volutes 802 are tangentially fed through openings 808 by a common liquid source within housing 806. The common liquid source is fed by the 1V458 Gear pump at 65 psig through an opening 808 that, although normally used as the discharge of a pump, is utilized as the input for the purpose of establishing a rotational vortex. The liquid entering the four volutes 802 is directed in a circle 360 degrees and discharged by the means of an 1″ long acceleration tube with a ⅜″ discharge hole. The discharge hole would normally be the suction side of a pump volute but, in this case, is utilized as the discharge side of the device. The four reverse fed volutes 808 establish rotational vortexes that spin the liquid through one 360 degree rotation, then discharge the liquid down the four acceleration tubes, each of which provides a 6 degree decreasing angle (as measured from the center line of the tube) acceleration section. The accelerated liquid is discharged into a common chamber 810 at or close to atmospheric pressure. The common chamber is connected to a stainless steel discharge line that feeds back into the top of a tank containing the liquid. At this point, the liquid has made one treatment pass through the device. The process described above is repeated continuously until the energy created by the implosions and explosions of the cavitation (e.g., due to the acoustical energy) have imparted sufficient kinetic heat to the liquid to raise the temperature to a desired level or until a specified processing period has expired. For water, the threshold temperature is about 60° C.

The same or a similar process whereby the liquid or liquids is/are subjected to one or more rotational vortices starting under reduced pressure and experiencing pressure gradients such that cavitation bubbles are formed and implode and explode through the process, will be referred to herein as “physics device”, and/or “physics process” , and/or “vortexing device”, and or “cavitation device”, and/or “cavitating process” and/or “fractionating device”.

An exemplary system for mixing of oil or particles in water is illustrated in FIG. 2. Liquid to be processed is introduced into the process loop through inlet port 240 in tank 216 and is pumped into cavitation device 100 by pump 202 through a 316 stainless steel line 208 to a Y-connection 210 which distributes the liquid to the two inlet ports 108, 110 or device 100. Alternatively, liquid or one component to be mixed into the liquid may be introduced through a cannula connected to the vacuum port 604 of one of the vacuum plates 306. The liquid is pumped into cavitation device 100 at a pressure such that rotational vortices are produced in each nozzle. The pressure will depend upon the type and viscosity of the liquid to be processed and the nozzle orifice sizes, but the pressure generally falls within the range of 55 to 150 psig. An exemplary pressure for processing water is 65 psig. After subjecting the liquid to the cavitation process, it leaves the device through discharge port 132 and is directed through stainless steel lines 212 and 214 into stainless steel tank 216. The liquid continues from tank 216 through stainless steel line 222 back to pump 202 for recirculating through the cavitation device for as many iterations until the desired termination point is achieved. Pressure gauge 204 measures the output pressure from pump 202 and digital temperature readout 206 displays the temperature of the liquid as it enters the cavitation device 100. During processing of water as described in the priority applications, the thermo-physical reactions that occur during the cavitation process cause the water temperature to increase. The temperature is permitted to rise and processing is deemed completed when the water temperature reaches a specified temperature. However, in certain processes, it may be desirable to control the rate of temperature increase in the fluid to maximize mixing time without allowing the fluid to become excessively heated. As illustrated, an optional temperature regulation unit 220, such as a heat exchanger, cooling jacket, or other cooling means as are known in the art, can be incorporated into the processing loop. While the temperature regulation unit 220 is illustrated downstream from the tank 216, it may be placed at other positions within the loop to achieve the same result. In another embodiment, a cooling jacket may be placed around tank 216.

In a preferred embodiment, temperature regulation is provided by cooling coils 242 that enter tank 216 through liquid tight ports in its base or sidewall. The coils should be positioned to avoid interference with the flow of liquid into and out of the tank. The coils are connected to a recirculating cooling bath 244 by tubing 246. Water or other coolant such as ethylene glycol is circulated though coils 242, the outer surfaces of which come into direct contact with the liquid within tank 216 to draw heat away from the liquid to provide temperature regulation. In the preferred embodiment, the coils 242 and tubing 246 are ½ inch copper tubing, which provides a significant advantage since the copper serves as a natural preservative. To enhance the preservative effect, a preferred process includes the addition of a small (catalytic) amount of ascorbic acid into the liquid being processed. The result of the reaction between the ascorbic acid and the copper is a neutral chelate that is naturally anti-fungal, anti-microbial, anti-viral and anti-inflammatory, such that these properties are imparted to the mixture that is being processed. As is known in the art, to provide the desired preservative effect, coils 242 may be formed from other metals that will form neutral chelates in the presence of an appropriate catalyst that is safe for inclusion in the fluid. Other metals include, but are not limited to silver, gold, zinc, platinum, tungsten, palladium, etc.

Once the desired processing has been completed, as determined either by time or by reaching a specified temperature threshold, valve 230 is opened to direct the processed liquid out of the loop through tubing 232 and into an appropriate storage vessel or other container(s) (not shown). While tubing 232 is illustrated as flexible tubing, it will be readily apparent that rigid tubing, such as the stainless steel line used elsewhere in the loop, may be used to provide a connection between the valve and a reservoir or tank through which liquid may be discharged from the loop.

As used herein, the term “micro-clustered composition” refers to a composition that comprises micro-cluster water. The adjective “micro-clustered” which modifies any of the compositions of bio-affecting agents, body-treating agents, adjuvant or carriers, or ingredients thereof refers to micro-clustered water in that composition, i.e. which is dissolved in, mixed with, or otherwise combined with micro-cluster water. A micro-cluster liquid is any liquid, mixture or combination of liquids, whether or not miscible, which has been processed according to the device described and claimed in U.S. Pat. No. 6,521,248.

The micro-cluster water produced by processing through the cavitation device has increased potential energy as compared with double distilled water. Although not intending to be bound by any proposed theory, it is believed that this increased energy allows the micro-cluster water to quench free-radicals and function as an anti-oxidant. The interaction of water and modified water media with various biological structures and processes is mainly determined by the unique role water plays in all biological systems. Water is a major constituent in most biological processes, as well as the fluid medium through which proteins and nucleic acids interact. Apart from being known as the main medium for biological reactions, water also plays a role in determining and stabilizing hydrophilic and lipophilic structures. Due to water's unique capabilities, it is able to influence the efficacy of various processes. However, many aspects related to the biological function of water remain unclear. There are facts, which indicate that the biological activity of water is due to a change in physical/chemical parameters. One of the important aspects in gaining an understanding of the mechanism controlling water's biological activity is to study it at the cell level. Water is highly related to the internal regulation system, including intracellular pH and cell membrane status.

A mixture of substances to be subjected to the cavitation device is processed in the same manner as water is processed through the device. As occurs during the processing of water, an increase in temperature is observed in the liquid mixture as it is processed. The resultant product is a substance (oil, particulate solid, or a combination thereof) dissolved in water or is water dissolved in oil, which are new compositions of matter.

The inventive device and the use thereof for the dissolving of lipophilics in hydrophilics, and/or the dissolving of hydrophilics in lipophilics, has broad and extensive applications, in the food, medical, cosmetic, environmental, manufacturing and pesticide industries. In any application where it is desired to increase solubility of a lipophilic substance in a hydrophilic liquid, or the inverse, the present inventive device and method are envisioned.

A general method applicable to many applications involves the combination of the substances to be mixed and otherwise dissolved into each other. Pre-mixing is not required. The composition is subjected to the cavitation device in an iterative manner until the desired temperature is achieved, which for olive oil and water is 140° C. Optimization of the preferred number of iterations may be performed without requiring undue experimentation.

It should be noted that the term “dissolve” is part of a continuum of mixtures. At one end is a pure substance. As one moves along the solvation line, component A is mixed with component B. Where there is true solvation, or miscibility, the atoms of component A are interdispersed with the atoms of component B. If components A and B are miscible, then there are mutually agreeable ionic interactions between all atoms. However, where A and B are not miscible, polar-non-polar interactions ensue and partial or complete separation of the components occurs. In a suspension or dispersion, small micelles are formed of one component that is dispersed or suspended in the other. This arrangement decreases the surface area of repulsive forces. The micelles may be of any size. As the size of the micelles that are suspended or dispersed in the solvent decreases, the system approaches a solvated system. Accordingly, within the context of the present application, a solvated system encompasses microparticulate suspensions and dispersions, whether lipophilic micelles in hydrophilic suspensate, or the inverse. The present application includes combinations of solvation, suspension and dispersion with one or more components, where one component may be miscible in one or more components of the mixture, but which form micelles and are suspended in another component. The types and forms of these mixtures are numerous and increase in complexity based on the number of components in the mixture. The present device and methods of mixing are directed to such complex mixtures.

The term “oil” should be broadly understood to include any lipophilic substance, including where one lipophilic substance is attached or associated with a more traditional oil. For example, a pharmaceutical compound may be bound or associated with an oil such as olive, cotton, linseed or similar, and is included under the terms and the scope of the claims. One or more than one oil shall be included in the term of oil, which is not limited to the singular, but shall include the plural without detracting from, nor limiting the scope of the claims. Moreover, where an oil or lipophilic substance has optical orientation, all enantiomers and diasteriomers and their isomeric derivatives are expressly envisioned.

Other lipophilic substances such as the non-oil perfumes and odorants may be processed in a manner similar to that of oils and shall be understood and included in this invention. Such organic substances are typically soluble in alcohols, yet when subjected to the present device, have increased water solubility.

As used herein, “metastable liquid” shall mean a liquid presenting one or more properties which are different as compared to a normal liquid. A normal liquid in this context shall mean a liquid not having modulated properties, under standard or known conditions, as disclosed in scientific literature and/or known to those of ordinary skill in the relevant art. A “micro-cluster liquid” shall also mean a metastable liquid.

All terms shall include customary and traditional meanings as well as additional interpretations provided by the documents previously incorporated by reference. Any ambiguous or vague term shall first be understood according to the context of the present document, with additional clarification provided according to the disclosures of the documents incorporated by reference.

In addition to the processing of lipophilic substances, the present invention is useful for processing of liquids of varying viscosity. Although not wishing to be bound by any particular theory, it is believed that the physics of the multi-rotational vortex through which the liquid is forced into laminar, rotating, sheet forming flow is an important aspect of the process. One of skill in the art will understand any necessary alterations to physical dimensions to provide such a result.

It has been found that familiar hydrophobic materials can be formed into stable aqueous dispersions by the application of an extraordinary high-pressure, high-shear process that utilizes unique blends of alkylated phosphatidyl choline (soy-derived lecithin). Molecules of phosphatidyl choline and certain other phospholipids will form assemblies with one another in water at extremely low concentrations with a low input of energy. These assemblies are typically bilayers with the polar head group of molecules interacting with aqueous phase. Concurrently, the non-polar, aliphatic portions of several molecules interact with one another or with the non-polar fluid to form a bi-layer.

Phosphatidyl choline can form up to eleven different stereo-chemical assemblies in water depending on the alkyl groups present, the phase transition temperature of the molecule, the concentration of the phosphatidyl choline present, the temperature at the time of formation, and the shearing energy applied during formation. Some of these assemblies are more thermodynamically stable than others depending on the systems energetic state during formation. Typically assemblies formed above the temperature at which the molecule changes the structural character of the phosphatidyl choline (i.e., transition temperature) are more stable because of the lower entropy present. However, assemblies often transition to a less stable assembly as the system is cooled. One type of more stable assembly is known as the lamellar phase (Lα). However, the Lα phase is difficult to form because it requires high energy, even extreme energies.

The solution to this problem is the introduction of high-energy input at low temperatures. This can be achieved by exposing phosphatidyl choline to extremely high shear rates under extreme pressure. One way that such shear can be achieved is by having a fluid physically diverted into two channels that impinge upon each other in a chamber at a substantial velocity, as occurs with the cavitation device. Similarly, extremely high shear rates under extreme pressure and temperature are achieved during the collapse of cavitation bubbles. Under the right combination of shear and pressure, enough energy can be imparted to allow almost instantaneous formation of extremely small droplets of the hydrophobic fluid, which are stabilized by concomitant formation of lamellar phase phosphatidyl choline assemblies. Since the formation process is almost instantaneous, the amount of time that the process media needs to be exposed to high shear rates and extremely high pressures can be very short. This time duration is so short, in fact, that the phosphatidyl choline assemblies formed do not have time to disassemble before they are no longer exposed to the shear and pressure conditions used to form them. Remarkably, by employing this procedure, lipophilic materials can be successfully incorporated into an otherwise all-water-based product.

This second type of assembly that can form is the result of a conversion that occurs in presence of relatively large amounts of hydrophobic material and water. Here, the phosphatidyl cholines rest at the surface of the hydrophobic droplets. The lipophilic tales of phosphatidyl choline extend into the hydrophobic droplets while the more polar heads of the phosphatidyl choline interact with the surrounding water to produce a micelle-like structure. Unlike many emulsions prepared by standard emulsification means, the amount of hydrophobe that can be accommodated into a stable, water miscible dispersion can be greater than 50% by weight. Different hydrophobes vary in their ability to be incorporated into the lamellar phase configuration. Generally, non-polar hydrophobes can be incorporated more easily than can more polar ones. The result of this process is a stable dispersion of highly concentrated hydrophobes that can, thereafter, be freely dispersed in water or water-based products without the risk of separation that occurs in most combinations of this type. Typically, the particle size of the micelle created during this process will be from 100 to 500 nanometers in diameter. This size is about one-tenth to one-fiftieth the size of particles produced by standard emulsification techniques.

The application of the cavitation process of the present invention to oil in water results in the formation of a microemulsion. The inclusion of a phosphatidyl choline, such as a soy-derived lecithin, results in the formation of micelle-like assemblies having a structure generally illustrated in FIG. 9.

The following examples illustrate the application of the cavitation device and method to mixing of various substances with water that has previously been processed using a cavitation device as described above and other steps (cooling and oxygenation) as described in U.S. Pat. No, 6,521,248. Such water is commercially available from Bio-Hydration Research Lab, Inc. (Carlsbad, Calif., USA) under the trademark Penta®.

The general procedure for mixing hydrophobic liquids in Penta® water is as follows: the oil or hydrophobic liquid is combined with phosphatidyl choline (soy-derived lecithin) and mixed at room temperature by agitation or stirring until a uniform mixture is achieved. The cavitation device is charged with an appropriate amount of Penta® water at room temperature, 70° F. (21° C.). The oil and lecithin solution is then added to the cavitation device. The mixture is circulated through the cavitation device and system (such as that illustrated in FIG. 2) until the desired particle size, and/or temperature and/or property are achieved.

### EXAMPLE 1

A mixture of 10% by volume Olive Oil in water is subjected to the cavitation process with test samples taken at 115°, 125°, 135° and 140° C. for determination of the amount of oil dissolved in the water. The maximum amount was 0.04 grams per 10 mL of water.

The oil in water solution has a milky white appearance with a faint Olive Oil odor. The solution was applied to the hands and arms as a lotion and was absorbed very rapidly into the skin and did not leave an oily film or feeling on the skin. The solution was subjected to centrifuge at 12,000 rpm for three 20 minute periods without causing a separation. The solution was further subjected to centrifuge at 20,000 rpm for three 20 minute periods, without separation or change in the solution.

### EXAMPLE 2

100 g of ZnO powder, 99.99% purity was combined with 20 1 of Penta® water and processed until the temperature reached 140° F. The calculated particle size, according to the Turbiscan™ device was between 0.04 μm and 0.012 μm.

### EXAMPLE 3

The device used to generate the oil in water according to Example 1 was further fitted on the exterior thereof with a cooling jacket or heat exchange system such that, as heat was generated, it was pulled away from the outside of the device, thereby maintaining the process temperature at a desired point. The system was allowed to process for two hours at 100° F. after which the heat exchanger was removed and the temperature of the liquid was allowed to increase to 140° F. after which the device was turned off and the processed oil in water collected. The average particle size was determined to be 115 nm.

### EXAMPLE 4

100 g of ZnO and 100 ml of olive oil were added to 20 L of Penta® water in a device similar to those used in the previous Examples. The ZnO had a particle size of 70-80 μm.

The device was also fitted with a temperature control means, such as described previously. The cavitating process was initiated and the internal mixture was allowed to increase in temperature to 100° F. whereupon the temperature control means was initiated and the temperature was held at about 100° F. for two hours. Thereafter, the temperature was allowed to increase to 140° F. whereupon the device was turned off and the mixture collected and analyzed. The ZnO was calculated to have a particle size range of between 0.04 μm and 0.012 μm and the olive oil was determined to have a particle size of 112 nm.

Through the use of the Turbiscan™ device, it has been determined that particles are produced that are typically smaller than 6 μm, and some are smaller than 180 nm. The observation of particles is important, as it tends to support the conclusion that micelle-like particles are being formed instead of micro-layering. Although the formation of micelle-like assemblies requires high energy, globally high temperatures and pressures are neither employed nor required. The localized energy produced by the collapse of cavitation bubbles is exploited for the needed temperature and pressure. Through the combined use of the cavitation device and Penta® water, oil in water systems comprising up to 50% oil by volume have been produced. The analyzed samples where stable under conditions described. Based on these results, it is believed that micelle-like structures are being formed through the described process.

The incorporation of other hydrophobic substances into the micelle pocket is further contemplated and well supported by these results. The incorporation of vitamins and other hydrophobic materials of biological importance are desirable, especially in view of the metabolic importance of phosphatidyl choline.

Although the examples demonstrate the method whereby the micelles are “filled” or loaded with the hydrophobic material coincident with their formation, i.e. in the cavitation device, it is also envisioned to generate “empty” micelles. Empty micelles are made through the same process as the filled ones, except that the process is run in the absence of a lipophilic component. In this manner, the micelles are formed but are empty, awaiting the introduction of a lipophilic material therein. These empty micelles are filled by high shear mixing with the desired hydrophobe. Such empty micelles may be referred to as “loadable” micelles and/or liposomes. It is not essential that these empty micelles be filled. The loadable micelles also function as non-detergent cleaners, perhaps by pulling the contaminant into the core of the micelle.

For the following examples, particle size analysis was performed using the Turbiscan™ device manufactured by Formulaction (France). This device relies on multiple light scattering technology to determine emulsion stability as well as particle size. The liquid mixtures are held in a transparent cell made of a borosilicate glass tube that has dimensions on the order of 12 mm diameter by 140 mm long. The bottom of the tube is sealed with a black Teflon® plug that absorbs the light. An optical head using infrared light (850 nm) vertically scans a height of 65 mm, recording the transmitted and backscattered light intensities. Intensities are a function of particle concentration, particle size and relative refractive index. The zero time corresponds to the completion of transfer of the liquid into the cell, when scanning first starts. Stability of the micelle-like particles was determined using the Turbiscan™ device set to the “fixed” scan mode. A fixed scan provides the ability to determine a change in particle size over time. Particle coalescence, particle sedimentation, particle creaming and other stability indicators are obtained through the fixed scan analysis mode. In all graphs “d” denotes particle size in μm (micrometer)

### EXAMPLE 5

One liter safflower oil, 50 milliliters macadamia nut oil, 500 milliliters borage oil, 400 milliliters lecithin were combined. Eight liters of Penta® water were added to the cavitation device and that device was put into operation mode. The hydrophobic mixture was added through standard means to the water in the device. Within one minute the water in the device developed a cloudy appearance, similar to milk. The temperature in the device was held at 110° F. (43.3° C.) for two hours using a temperature regulation unit as previously described.

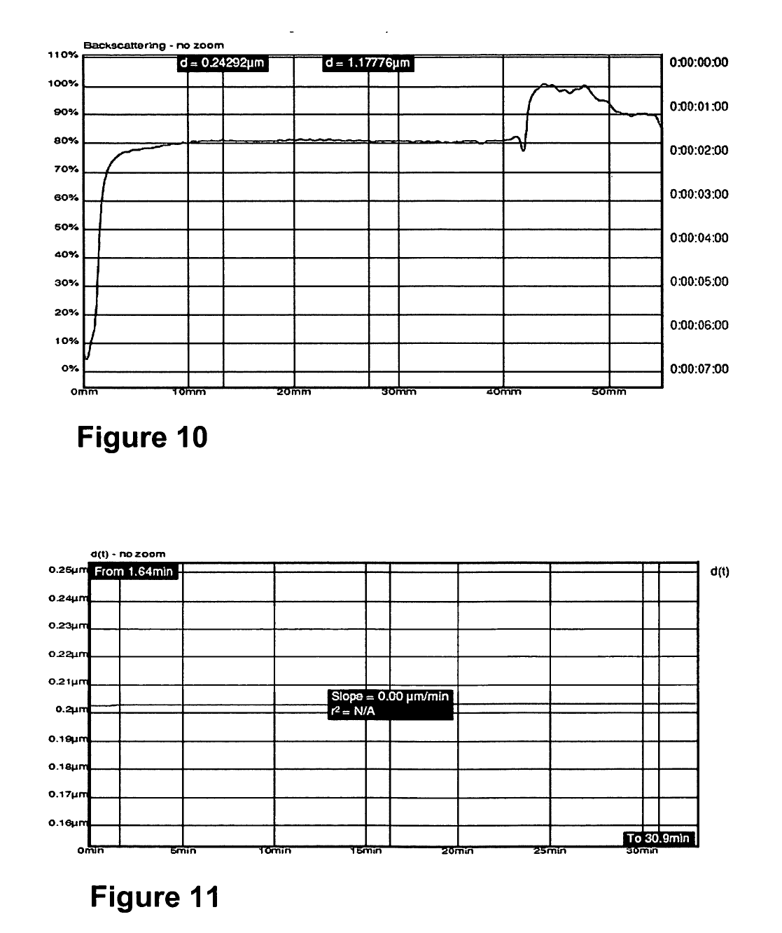

FIG. 10 illustrates the results of stability analysis of the mixture as measured using optical backscattering in a container of the liquid. As indicated by the single curve for multiple measurements with time, the solution is highly stable, with the percentage of backscattered intensity remaining constant at around 80% for liquid depths from about 8 mm from the bottom of the container up to about 42 mm. Particle diameter at about 13 mm depth was measured as 0.24292 μm and was 7776 μm at about 27 mm depth.

### EXAMPLE 6

Olive Oil 10%:

One liter olive oil combined with 473 milliliters lecithin. Eight liters of Penta® water where added to cavitation device and the device was put in operation mode. The hydrophobic mixture was added through standard means to the water in the device. Within one minute the water in the device became cloudy similar to milk. The temperature in the device was held at 110° F. (43.3° C.) for two hours. A sample was analyzed for stability. The results of the analysis are provided in FIG. 11, which is a plot of particle size versus time. As indicated, the particle size remained at about 0.202 μm throughout the 30.9 minute test, indicating a highly stable solution.

### EXAMPLE 7

Olive oil 50%:

Five liters olive oil combined with 833 milliliters lecithin. The device was charged with four liters Penta® (water and put in operation mode. The hydrophobic mixture was added through standard means to the water in device. Within one minute the mixture in the device developed a cloudy appearance, similar to milk, and within five minutes the mixture was thick, with a consistency similar toe butter. The device is turned off and samples collected. This material was too thick to ensure proper loading of the sample vial, therefore, no particle or stability analysis performed.

### EXAMPLE 8

Olive oil mixture:

One liter olive oil, 20 grams vitamin E, 15 grams steroyl ester, 40 milliliters Clarins™ tonic oil, 30 milliliters tea tree oil, 250 milliliters grape seed oil combined with 400 grams lecithin. Eight liters of Penta® water where added to cavitation device and the device was put in operation mode. The hydrophobic mixture was added through standard means to the water in the device. Within one minute the mixture in the device developed a cloudy appearance, similar to milk. The temperature in the device was held at 110° F. (43.3° C.) for two hours using a temperature regulation unit as previously described.

### EXAMPLE 9

Jojoba oil:

16 ounces (473 milliliters) jojoba oil was combined with 96 milliliters lecithin. The device was charged with eight liters of Penta® (water and put in operation mode. The oil and lecithin solution was added through standard means to the water in the device. Within one minute the mixture in the device developed a milky appearance. The device was turned off after thirteen minutes, at which point the temperature was 115° F. (46.1° C.) and samples taken. A stability analysis was performed.

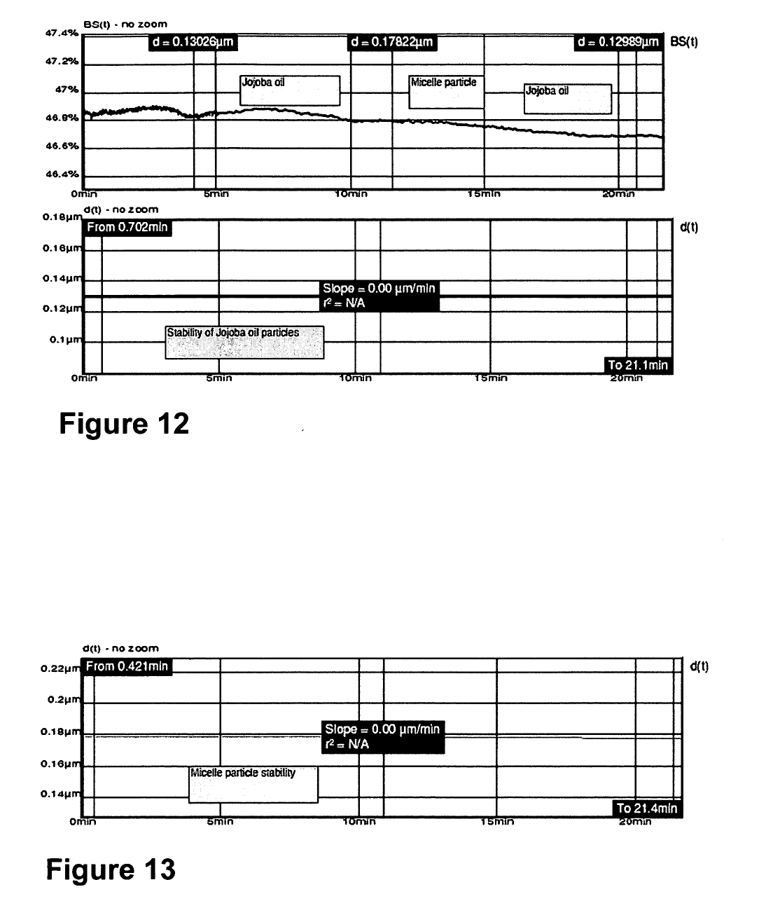

The results of the analysis are shown in FIG. 12, where the upper plot shows percent backscatter with time, up to just over 21 minutes for both jojoba oil and lecithin particles. As indicated, the backscatter decreased very slightly, from about 46.93% to 46.72% over the testing period. The diameter of the jojoba oil particles varied from 0.13026 μm at about 4.5 minutes to 0.12989 μm at 21 minutes into the test. The lecithin particle diameter was measured as 0.17822 μm at around 12 minutes into the test. The lower plot in FIG. 12 provides changes in particle diameter with time. The slope of 0.00 μm/min demonstrates excellent particle size stability at around 0.130 μm. A similar analysis was performed with a focus on the phosphatidyl choline particles. The results of this analysis are provided in FIG. 13. As in the measurement of the jojoba oil particles, the zero slope indicates no change in particle size with time.

### EXAMPLE 10

Tea Tree oil:

16 ounces (473 milliliters) Tea Tree oil was combined with 96 milliliters lecithin. The device was charged with eight liters of Penta water and put in operation mode. The oil and lecithin solution was added through standard means to the water in the device. Within one minute the mixture in the device developed a milky appearance. The device was turned off after thirteen minutes and samples taken.

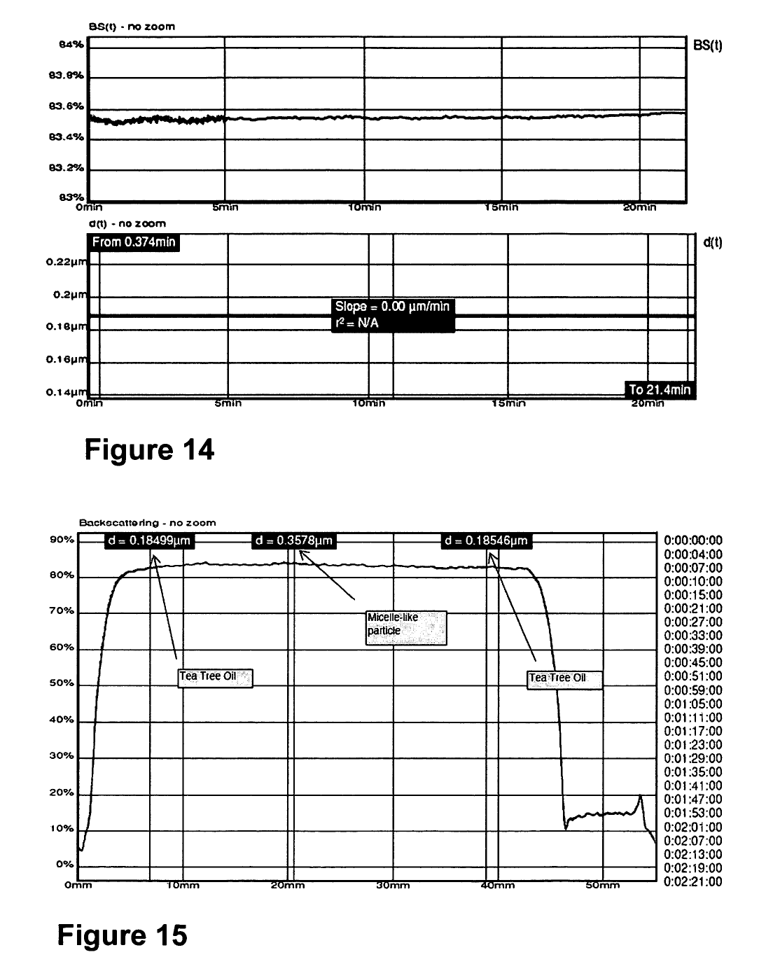

A sample was analyzed for stability. The results are provided in FIGS. 14 and 15. In FIG. 14, the upper plot shows backscatter percentage with time, up to just over 21 minutes. As indicated, the high level of backscatter remained constant over the testing period, varying less than 0.1%. The lower plot in FIG. 14 provides changes in particle diameter with time. The slope of 0.00 μm/min demonstrates excellent particle size stability at around 0.185 μm.

FIG. 15 is a plot of backscatter percentage versus depth within the sample cell over a time period of 2 minutes, 21 seconds. As indicated by the overlapping curves, the sample was very stable over the test period. The sample had uniform backscatter of over 80% from a depth of about 5 mm up to about 45 mm, dropping off sharply to around 15% at 50 mm. The diameters of the tea tree oil particles were very uniform as well as uniformly distributed, varying from 0.18499 μm at about 7 mm depth to 0.18546 μm at about 38 mm. The lecithin (micelle-like) particle diameter was measured as 0.3578 μm at a depth of about 21 mm.

### EXAMPLE 11

The oil contained in micelles is often less viscous than water. A 10% olive oil solution in 1% micelles was placed in a misting spray applicator, as is known in the art. Such a solution provides for the application of a moisturizing lotion using a convenient spray technique.

### EXAMPLE 12

The oil contained in micelles are fully and freely miscible with water at all proportions. Once the micelles are formed, they are water soluble at all proportions. A 30% Tea Tree oil in 1% phosphatidyl choline micellular system was produced through the cavitation device as described above in Example 10. One ml of this solution was added to 100 ml of Penta® water and, with only minor agitation, a uniform solution was obtained.

### EXAMPLE 13

The oil contained in micelles are fully and freely miscible with water at all proportions. Once the micelles are formed, they are water soluble at all proportions. 150 ml of a 30% Borage oil in 10% phosphatidyl choline micellular system was produced through the cavitation device as described above. This solution was added to a bathtub containing about 35 gallons of tap water. The Borage oil micellular system dispersed instantly, changing the bath water to a uniform milky white consistency. The Borage oil-micelles were attracted to the skin of the bather and without sticking to the sides of the bathtub.

### EXAMPLE 14

The oil contained in micelles are fully and freely miscible with water at all proportions. Once the micelles are formed, they are water soluble at all proportions. A 30% jojoba oil in 12% phosphatidyl choline micellular system was produced through the cavitation device as described above. This mixture was placed in a misting spray applicator. This solution was applied just after either bathing or showering, while the skin is still wet. The oil-micelle system instantly dissolved in the water droplets on the skin and was quickly absorbed into the skin.

The foregoing examples provide illustrations of the mixing of oils, or particulate solids, and water using the cavitation device to form stable solutions that are useful in cosmetic and medicinal agents, and in the manufacture of foods and chemicals, among other applications. Once the solution is formed using the cavitation device, it can be mixed with additional components, including water, to produce further uniform (non-layered) solutions simply by adding the cavitation device-produced solution to the additional components using conventional mixing techniques such as shaking or agitation, without additional processing through the cavitation device.

As used herein, the term “cosmetic agent” means any compound, mixture of compounds, or preparations derived therefrom that are intended to be placed in contact with external parts or with mucosal membranes of an animal body. (Especially a human body) with a view to cleaning, changing the appearance, protecting and/or keeping the body parts to which the agent is applied in good condition.

Preferably, the cosmetics agent is capable of diminishing, reducing or preventing the effects of one or more skin conditions including: the visible effects of aging, wrinkles, acne, age spots, scars (keloids) broken capillaries and, includes compositions which also optionally cleanse the skin, preferably in the form of liquid compositions such as liquid soaps, lotions and solutions both additives and compositions for application to skin, hair, scalp, nails, eyes or teeth.

Cosmetic agents include those that may be used in mesotherapy, which involves the injection of chemicals, vitamins or other materials into the mesoderm (fat layer) just under the skin, to treat various conditions in subcutaneous fat to reduce fat deposits or to minimize the bumpy appearance of the skin caused by cellulite. Mesotherapy is not limited to cosmetic applications, and may include medical uses such as treatment of bruises, allergic reactions, or infections.

The term “topical administration” includes methods of delivery such as laying on or spreading on the skin. It involves any form of administration, which involves the skin. Examples of compositions suitable for topical administration include but are not limited to, ointments, lotions, creams, cosmetic formulations, and skin cleansing formulations. Additional examples include aerosols, solids (such as bar soaps) and gels.

Various vitamins and minerals may also be included in the mixtures produced using the present invention. For example, Vitamin A, ascorbic acid, Vitamin B, biotin, panthothenic acid, Vitamin D, Vitamin E and mixtures thereof and derivatives thereof are contemplated.

Sunblocks and sunscreens incorporating micro-cluster liquids and creatine compounds are also contemplated. The term “sun block” or “sun screen” includes compositions, which block UV light. Examples of sunblocks include, for example, zinc oxide and titanium dioxide, which are mixed in water and/or lipophilics as previously described.

Sun radiation is one major cause of skin damage, e.g., wrinkles. Thus, for purposes of wrinkle treatment or prevention, the combination of a micro-cluster liquid and a creatine compound with a UVA and/or UVB sunscreen would be advantageous. The inclusion of sunscreens in compositions of the present invention will provide immediate protection against acute UV damage. Thus, the sunscreen will prevent further skin damage caused by UV radiation, while the compounds of the invention modulates existing skin damage.

Preferred sunscreens useful in the compositions of the present invention are nanometer particles of TiO₂, ZnO, dispersed in a micro-cluster liquid and mixtures thereof.

A safe and effective amount of sunscreen may be used in the compositions of the present invention. The sunscreening agent must be compatible with the active compound. Generally the composition may comprise from about 1% to about 20%, preferably from about 2% to about 10%, of a sunscreening agent. Exact amounts will vary depending upon the sunscreen chosen and the desired Sun Protection Factor (SPF).

Although the present invention has been described herein with reference to particular means, materials, and embodiments, the present invention is not intended to be limited to the particulars disclosed herein; rather, the present invention extends to all functionally equivalent structures, methods and uses, such as are within the scope of the appended claims.

1. A system for mixing a liquid with at least one substance comprising a lipophilic or a particulate solid, comprising:
a cavitation device comprising:
a housing having a pair of liquid inlets for introducing liquid into the device;
a pair of axially aligned nozzles disposed within the housing, each nozzle corresponding to a liquid inlet and having a tangential inlet channel and a conical interior surface for receiving the liquid under pressure and generating a rotational vortex for spinning the liquid in a circle and directing the liquid through an exit orifice into a common chamber disposed at a center of the housing between the nozzles, wherein the rotational vortex creates a partial vacuum within the spinning liquid so that cavitational energy is produced when the liquid exits the nozzle, and wherein the nozzles are disposed with their exit orifices opposing each other so that liquid emitted from the nozzles collides so that the liquid exiting from the nozzles generates additional cavitational energy for mixing the liquid; and
a discharge line connected to the common chamber for discharging the liquid from the housing;
a processing loop for recirculating the liquid through the cavitation device until one or more selected criteria for a mixed liquid are met;
a pump for feeding the liquid into the pair of inlets at a first pressure;
at least one inlet port for introducing the liquid into the system; and
an outlet port for removing the mixed liquid from the loop;

2. The system according to claim 1, wherein the nozzles are slidably disposed within the housing so that a distance between the exit orifices of the nozzles is adjustable.

3. The system according to claim 1, further comprising a temperature regulation unit for drawing heat from the liquid.

4. The system according to claim 3, wherein the temperature regulation unit comprises a coolant recirculator and a cooling coil, wherein the cooling coil is disposed within the loop in direct contact with the liquid.

5. The system according to claim 4, wherein the cooling coil is formed from a metal that forms a neutral chelate that is a natural preservative.

6. The system according to claim 5, wherein the coolant coil is formed from copper.

7. The system according to claim 1 wherein the liquid emitted from the nozzle is a rotating laminar flow to form a hollow cone.

8. A method for mixing a liquid and an oil or particulate solids comprising:
processing the liquid in a processing loop including a pump and a cavitation device comprising:
a housing having a pair of liquid inlets for introducing liquid into the device;
a pair of axially aligned nozzles disposed within the housing, each nozzle corresponding to a liquid inlet and having a tangential inlet channel and a conical interior surface for receiving the liquid under pressure and generating a rotational vortex for spinning the liquid in a circle and directing the liquid through an exit orifice into a common chamber disposed at a center of the housing between the nozzles, wherein the rotational vortex creates a partial vacuum within the spinning liquid so that cavitational energy is produced when the liquid exits the nozzle, and wherein the nozzles are disposed with their exit orifices opposing each other so that liquid emitted from the nozzles collides so that the liquid exiting from the nozzles generates additional cavitational energy for mixing the liquid; and
a discharge line connected to the common chamber for discharging the liquid from the housing;
discharging the liquid into the loop to for recirculating through the cavitation device;
repeating the step of processing until one or more pre-determined criteria are met; and
after the pre-determined criteria are met, discharging the liquid from the loop.

9. The method of claim 8, further comprising regulating a temperature of the liquid within the loop.

10. The method of claim 9, wherein regulating a temperature comprised placing a cooling coil in direct contact with the liquid and recirculating a coolant through the cooling coil to draw heat from the liquid.

11. The method of claim 10, wherein the cooling coil is formed from a metal that forms a neutral chelate that is a natural preservative.

12. The method of claim 11, further comprising adding a catalyst to the liquid to enhance formation of the neutral chelate.

13. The method of claim 12, wherein the metal is copper and the catalyst is ascorbic acid.

14. The method of claim 8, further comprising varying an axial separation between the nozzles according to a viscosity of the liquid.

15. The method of claim 8, wherein the one or more pre-determined criteria comprises a fixed processing period.

16. The method of claim 8, wherein the liquid is water and the oil is hydrophobic and further comprising adding phosphatidyl choline to the oil prior to processing.
